(19) United States
(12) Patent Application Publication
(10) Pub. No.: US 2013/0143292 A1
(43) Pub. Date: Jun. 6, 2013

(54) DEVICE AND METHOD FOR THE ANAEROBIC DIGESTION OF ORGANIC MATERIAL TO BIOGAS BY MEANS OF MICRO-ORGANISMS

(71) Applicant: RED PATENT B.V., Gorssel (NL)

(72) Inventor: Jacob Hendrik Obbo HAZEWINKEL, ZOETERMEER (NL)

(73) Assignee: RED PATENT B.V., GORSSEL (NL)

(21) Appl. No.: 13/745,911

(22) Filed: Jan. 21, 2013

Related U.S. Application Data
(63) Continuation of application No. PCT/NL2010/000112, filed on Jul. 19, 2010.

(51) Int. Cl. C12M 1/107

(52) U.S. Cl. CPC C12M 21/04
USPC 435/167; 435/290.2

(57) ABSTRACT

The invention relates to a device and method for the anaerobic digestion of organic material to biogas by means of microorganisms. The device and method intensify the anaerobic digestion process by using three separate reactors wherein a hydrolysis process, a first digestion process of a separated solid fraction, and a second digestion process of a separated liquid fraction take place respectively. Membranes and/or mechanical separation means are used to separate the fractions. This keeps the microorganisms, such as microbes, in the reactors. The obtained intensification of the anaerobic digestion process enables to operate with smaller tanks, which leads to lower building costs. The process also makes it possible to precipitate heavy metals for extraction.

### CROSS REFERENCE TO RELATED APPLICATIONS

This application is a continuation of PCT application number PCT/NL2010/000112 filed on 19 Jul. 2010, this application is hereby incorporated by reference in its entirety.

### BACKGROUND OF THE INVENTION

1. Field of the Invention

The invention relates a device for the anaerobic digestion of organic material to biogas by means of microorganisms. The invention further relates to a method for the anaerobic digestion of organic material to biogas by means of microorganisms, particularly using the above mentioned device.

2. Description of the Related Art

The major objective of anaerobic digestion of organic material is the production of biogas. In anaerobic digestion, organic material, for example animal fat and garden waste, is broken down to smaller molecules, such as for instance methane and carbon dioxide, in a number of different stages, substantially in the absence of air or any other source of oxygen.

The first stage of anaerobic digestion of a mass of organic material typically comprises hydrolysis of the organic material. In hydrolysis, the larger organic molecules, such as proteins, carbohydrates and fats are broken down by microbes to smaller molecules, such as acids. A drawback is that hydrolysis of for instance lignocellulosis by microbes is slow. Lignocellulosis is a typical component of the solid residues of anaerobic (and aerobic) digestion.

Hydrolysis usually yields a substantially liquid fraction, comprising a mixture of acids and other organic molecules in a more or less liquid state, and a substantially solid fraction, comprising for instance undigested organic material, such as lignocellulosic material.

The liquid fraction of the liquid/solid mixture is then typically converted by microbes into smaller organic molecules, such as organic acids and alcohols, and finally into methane and carbon dioxide. The latter process, known as methanogenesis, is a relatively slow process, and therefore needs relatively large tanks to make the process economically viable. Such large tanks however require a large investment cost.

Most devices for anaerobic digestion known today comprise one reactor tank, wherein all stages of anaerobic digestion take place. Due to the significant hold up time to obtain sufficient digestion, the tanks are of a significant size, hence influencing the building costs. This effects the economic viability of biogas production from organic material. In some systems, the hydrolysis process takes place in a separate tank, as a kind of preprocessing stage, under different conditions than in the actual anaerobic digestion stage, in order to decrease the time of conversion and therefore decrease the size and cost of the tanks. An example of such a method and device is given in U.S. Pat. No. 5,630,942.

It would be desirable to intensify the anaerobic digestion process and thereby increase bacterial activity. The size of tanks could then be decreased which lowers production cost. Several solutions have been proposed. The digestion process can for instance be intensified by keeping the amount of microbes in the digestion tank at a relatively high level. To this end, some known systems have a feed back of microbes in the liquid fraction after this fraction has been separated from the solid fraction, as described in JP 2008086869 and U.S. Pat. No. 4,491,522 for instance.

Another known method to intensify the anaerobic digestion processes is to separate the hydrolyzed liquid fraction from the solid fraction, followed by anaerobic digestion of the liquid fraction under conditions that are optimal and different from the anaerobic digestion process of the liquid/solid mixture. Such a method is described in GB 2407088 and WO 2007/015098 for instance. In both publications, microbes are returned with a part of the digested liquid fraction to a first phase of anaerobic digestion, in order to intensify this process.

JP 11147098 describes the separation of anaerobically digested material into a liquid and solid fraction by a membrane. After anaerobic digestion, the material is lead to a tank, wherein the material is separated in a liquid and solid fraction. The solid fraction is sent back to the anaerobic digestion process, in order to stimulate this process by adding microbes.

### BRIEF SUMMARY OF THE INVENTION

It is an object of the invention to provide an improved device and method for the anaerobic digestion of organic material to biogas by means of micro-organisms.

This and other objects can be achieved according to various aspects of the present invention by providing a device for the anaerobic digestion of organic material to biogas by micro-organisms, comprising a hydrolysis compartment for the hydrolyzation of organic material, a first separator for the separation of the product flow originating from the hydrolysis compartment in a substantially solid fraction and in a substantially liquid fraction, a first anaerobic digestion compartment for the digestion by micro-organisms of the substantially solid fraction to a gaseous fraction, a second anaerobic digestion compartment for the digestion micro-organisms of the substantially liquid fraction, and a second separator for the separation of the product flow originating from the second anaerobic digesting compartment in a liquid fraction substantially ions containing and biogas, the first and/or the separator being adapted for retaining the micro-organisms present in the first and/or second anaerobic digestion compartment.

This and other objections can also be achieved according to various aspects of the present invention by the method for the anaerobic digestion of organic material to biogas by micro-organisms, said method comprising the steps of: leading a mass of organic material into a hydrolysis compartment, at least partially hydrolyzing the mass of organic material in the hydrolysis compartment, leading at least a part of the hydrolyzed mass to the first separator, and separating the mass originating from the hydrolysis compartment by the first separator in a substantially solid fraction and a substantially liquid fraction, leading at least a part of the substantially solid fraction into the first anaerobic digestion compartment, and digesting the substantially solid fraction in the first anaerobic digestion compartment by the micro-organisms, leading at least a part of the substantially liquid fraction into the second anaerobic digestion compartment, and digesting the substantially liquid fraction in the second anaerobic digestion compartment by the micro-organisms, leading at least a part of the liquid fraction originating from the second anaerobic digestion compartment into the second separation means, and separating by the second separator the liquid fraction originating from the second anaerobic digestion compartment in a substantially ions-containing (ionic) liquid fraction and biogas, the first and/or the second separator being adapted for substantially retaining the micro-organisms present in the first and/or second anaerobic digestion compartment.

In summary, according to the invention a device and method for the anaerobic digestion of organic material to biogas by means of micro-organisms is provided which in one embodiment intensifies the anaerobic material to biogas by means of microorganisms is provided which in one embodiment intensifies the anaerobic digestion process by using three separate reactors wherein a hydrolysis process, a first digestion process of a separate solid fraction, and a second digestion process of a separated liquid fraction take place respectively. Membranes and/or mechanical separation means are used to separate the fractions. This keeps the micro-organisms such as microbes, in the reactors. The obtained intensification of the anaerobic digestion process enables to operate with smaller tanks, which leads to lower building costs. The process also makes it possible to precipitate heavy metals for extraction.

### BRIEF DESCRIPTION OF THE DRAWINGS

The features and advantages of the invention will be appreciated upon reference to the following drawings, in which:

FIG. 1 shows a schematic view of one embodiment of a device according to the invention; and

FIG. 2 shows a schematic view of another embodiment of a device according to the invention.

### DESCRIPTION OF ILLUSTRATIVE EMBODIMENTS

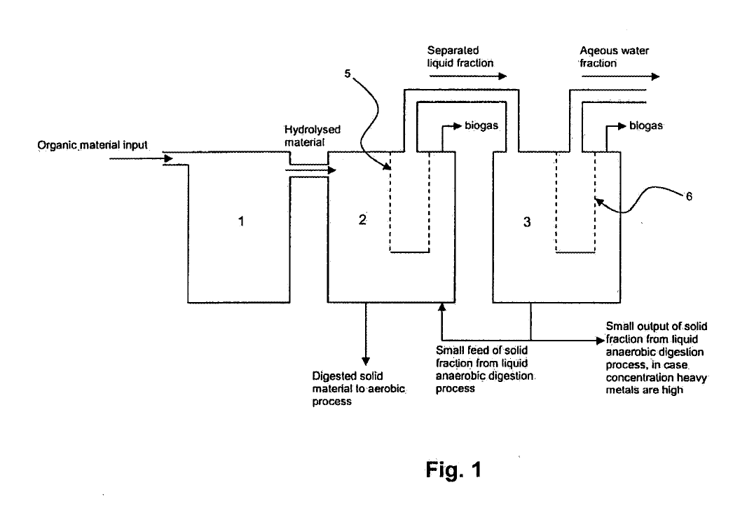

The following is a description of certain embodiments of the invention, given by way of example only and with reference to the drawings. Referring now to FIG. 1, a first reactor is shown. In reactor 1, a first stage of the anaerobic digestion process is carried out, i.e. hydrolysis. The acidity in the reactor 1 is preferably at a pH-value ranging from 3 to 5, and most preferably about 4. To enhance the hydrolysis process, the surface area of the mass of organic material is preferably enlarged by decreasing the particle size. Preferably the mass of organic material is thereto led to a macerator (not shown) before entering reactor 1. In hydrolysis reactor 1, large molecules of the organic material, such as proteins, carbohydrates, and fats are converted into smaller molecules, such as acids, that are soluble or form an emulsion (the liquid fraction) and are therefore separable from a solid fraction. The hydrolyzed mixture of liquid emulsion and solid fraction is then transported to a second reactor, the solid fraction anaerobic digestion reactor 2. This presents loss of microbes for the digestion process carried out in reactor 2, hence keeping the microbe population at a high level for a more intensified anaerobic digestion of the remaining solid fraction, and preventing the microbes of this reactor 2 from flowing into the third reactor 3. If the microbes of the reactor 2 for solid fraction anaerobic digestion would flow to the liquid fraction anaerobic digestion reactor 3 the methanogenesis process carried out in reactor 3 would be sub-optimal. Reactor 2 also functions to digest the remaining solid fraction, producing a small amount of biogas. The remaining solid fraction residue is finally led to an aerobic process for the production of compost for instance via the exit, as shown in FIG. 1.

In the liquid fraction anaerobic digestion reactor 3, the liquid fraction containing organic material in solution or emulsion, is anaerobically digested in so-called methanogenesis process, known per se. In this process, biogas is formed. Sludge that is formed in this reactor 3 is preferably transported back to the reactor 2 for solid fraction anaerobic digestion for further conversion. Water in which ions such as potassium, sodium, sulphate, magnesium and phosphate are dissolved, is led via a membrane 6 out of reactor 3.

Characteristic of the present invention is the use of a membrane 6 in reactor 3 to prevent loss of microbes for this digestion stage, hence maintaining the microbe population at a high level for a more intensified anaerobic digestion of the liquid fraction . Due to the intensification of the anaerobic digestion, the reactor tank 3 can be designed smaller, decreasing building costs. The same advantage is obtainable for the other reactor tanks 1 and 2.

The acidity in reactor 3 during digestion of the liquid fraction is preferably between a pH-value of 7 to 8, more preferably of about 7.3. The preferred pH range prevents the minerals from precipitating into crystals, such as for example struvite. Heavy metal salts, however, such as for instance copper, lead and/or zinc, precipitate at this level of acidity. Most of these heavy metals were in the soluble state in the hydrolysis reactor 1 and the reactor 2 for solid fraction anaerobic digestion, due to the typical level of acidity in these reactors, and therefore are retrieved in a substantially concentrated state in the solid fraction of the third reactor. According to a preferred embodiment of the invention, these precipitated salts are lead with the solid fraction particles back to the reactor 2 for solid fraction anaerobic digestion, in which reactor 2 they will finally be a part of the output of this reactor 2, which is used for the production of compost by an aerobic process. In case the level of heavy metals is higher than required for certain quality standards, this amount of solid particles in reactor 3 is lead to a separate tank and regarded as final waste.

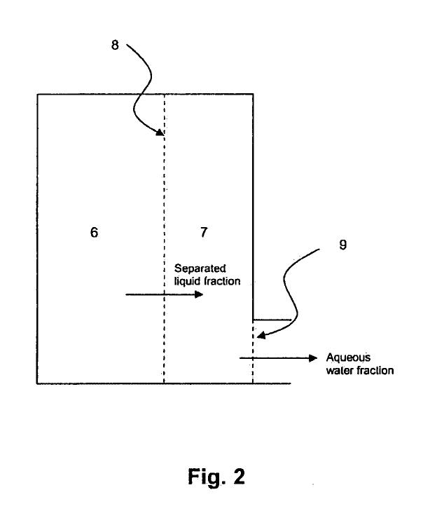

The above described digestion method can be operated both as a continuous process and as a batch process. An embodiment describing a batch process is shown in FIG. 2. As shown, one tank is provided with two membranes (8, 9). The tank is divided in two compartments (6,7) by a membrane 8. The batch process in the tank starts in the first compartment 6 in which the hydrolysis process step is carried out, followed by solid fraction anaerobic digestion. The liquid in the solid fraction is forced by hydrolytic pressure or any other applied pressure through the first membrane 8 to the second compartment 7 of the tank, here a liquid fraction anaerobic digestion is carried out at a pH-value preferably ranging from 7 to 8, and more preferable at about 7.3. By the action of hydraulic pressure or any other applied form of pressure, the liquid fraction is further forced through the second membrane 9 and removed from the tank as an aqueous solution containing at soluble struvite, potassium and/or nitrogen. The residue in the second compartment 7 of the tank is a sludge, comprising the substantially entire microbe population (for compartment 7), and precipitated heavy metal salts, such as for example copper, lead and/or zinc.

Characteristic of the present invention is the use of either a membrane 4 and/or mechanical separation means within the reactor tank 2 to separate the liquid fraction from the solid fraction that are formed after hydrolysis in the anaerobic digestion process, while the solid fraction is still being anaerobically digested and produces biogas.

The purpose of this separation is to keep the microbes substantially with the solid fraction, in order to maintain a high amount of microbes for intensification of the anaerobic digestion process. Intensification of the anaerobic digestion process allows and/or requires a smaller reactor tank, which significantly lowers the building costs.

Characteristic of the present invention is the use of a membrane 5 to separate the water fraction containing ions, from the liquid fraction in the reactor 3 of the anaerobic digestion process of the liquid fraction. The purpose of this separation is to keep the microbes substantially within this reactor 3, in order to maintain a high amount of microbes for intensification of the anaerobic digestion process of the liquid fraction. Intensification of the anaerobic digestion process allows and/or requires a smaller reactor tank, which significantly lowers the building costs.

A further characteristic of the present invention is that a concentration of heavy metal ions, such as for example copper, lead and zinc is obtained in the solid fraction of the third reactor, hence giving the possibility to isolate this fraction from other solid output of the process.

According to the present invention, it is possible to operate the describes method batchwise in a tank with two membranes (8, 9). The first membrane 8 forms a compartment 6 where the hydrolysis and the solid fraction anaerobic digestion takes place. In the second compartment 7 between the first 8 and second 9 membrane the liquid fraction anaerobic digestion takes place. The purpose of the first and second membrane is to keep the microbes with the solid fraction and liquid fraction respectively, in order to maintain a high amount of microbes for intensification of the anaerobic digestion process.

This invention is provides a process aimed to intensify the anaerobic digestion process by using three different reactors (1, 2 and 3) where mentioned processes take place and by using membranes (4 and 5) to separate fractions while keeping microbes in the reactors. By keeping the microbes in the reactor tanks the microbe population is kept at a high level, which maintains the intensity of the process at a high level. The goal of intensification of the anaerobic digestion process is to have the same throughput and biogas production with smaller thanks, which leads to lower investment costs.

Further modifications in addition to those described above may be made to the structures and techniques described herein without departing from the spirit and scope of the invention. Accordingly, although specific embodiments have been described, these are examples only and are not limiting upon the scope of the invention.

What is claimed is:

1. A device for the anaerobic digestion of organic material to biogas by micro-organisms, comprising a hydrolysis compartment for the hydrolyzation of organic material,
a first separator for the separation of the product flow originating from the hydrolysis,
compartment in a substantially solid fraction and in a substantially liquid fraction,
a first anaerobic digestion compartment for the digestion by microorganisms of the substantially solid fraction to a gaseous fraction,
a second anaerobic digestion compartment for the digestion micro-organisms of the substantially liquid fraction, and
a second separator for the separation of the product flow originating from the second anaerobic digesting compartment in a substantially ionic liquid fraction and biogas,
the first and/or the separator being adapted for retaining the micro-organisms present in the first and/or second anaerobic digestion compartment.

2. The device of claim 1, wherein the first and/or the second separator comprise a membrane and/or mechanical separation mechanism.

3. The device of claim 1, the first anaerobic digestion compartment comprises the first separator.

4. The device of claim 1, wherein the second anaerobic digestion compartment comprises the second separator.

5. The device of claim 1, wherein the device comprises a grinding, macerating or softening device arranged upstream relative to the hydrolysis compartment.

6. The device of claim 1, wherein the first anaerobic digestion compartment is provided with a discharge for the solid residue of the anaerobic digestion en/or the gaseous residue of the anaerobic digestion.

7. The device of claim 1, wherein the second anaerobic digestion compartment is provided with a discharge for the liquid residue of the anaerobic digestion en/or the gaseous residue of the anaerobic digestion.

8. The device of claim 1, wherein the second anaerobic digestion compartment is provided with a discharge for the solid residue of the anaerobic digestion, said discharge being connected with the inlet of the first anaerobic digestion compartment for the purpose of leading at least a part of the solid fraction from the second anaerobic digestion compartment to the first anaerobic digestion compartment.

9. A method for the anaerobic digestion of organic material to biogas by microorganisms, said method comprising the steps of:
leading a mass of organic material into a hydrolysis compartment,
at least partially hydrolyzing the mass of organic material in the hydrolysis compartment,
leading at least a part of the hydrolyzed mass to the first separator, and separating the mass originating from the hydrolysis compartment by the first separator in a substantially solid fraction and a substantially liquid fraction,
leading at least a part of the substantially solid fraction into the first anaerobic digestion compartment, and digesting the substantially solid fraction in the first anaerobic digestion compartment by the microorganisms,
leading at least a part of the substantially liquid fraction into the second anaerobic digestion compartment, and digesting the substantially liquid fraction in the second anaerobic digestion compartment by the microorganisms,
leading at least a part of the liquid fraction originating from the second anaerobic digestion compartment into the second separator, and separating by the second separator the liquid fraction originating from the second anaerobic digestion compartment in a substantially ionic liquid fraction and biogas,
the first and/or the second separator being adapted for substantially retaining the micro-organisms present in the first and/or second anaerobic digestion compartment.

10. The method of claim 9, wherein the first and/or the second separator comprise a membrane and/or mechanical separator mechanism.

11. The method of claim 9, wherein the performing of at least the partial hydrolyzation in the hydrolyzation compartment of the mass of organic material at a pH-value between 3 and 5.

12. The method of claim 9, wherein the grinding, macerating or softening of the mass of organic material preceding the hydrolyzation.

13. The method of claim 9, wherein the performing of the digestion of the substantially liquid fraction in the second anaerobic digestion compartment by the microorganisms at a pH-value of between 7 and 8.

14. The method of claim 13, wherein the pH-value is between about 7.2 and 7.4.

15. The method of claim 9, wherein digesting the substantially liquid fraction in the second anaerobic digestion compartment by the microorganisms, following separation by the second separator, gives a separated solid fraction of which at least a part is led back via the inlet of the first anaerobic digestion compartment.
